(19) United States
(12) Patent Application Publication
(10) Pub. No.: US 2015/0087898 A1
(43) Pub. Date: Mar. 26, 2015

(54) CAPSULE ENDOSCOPE MAGNETIC CONTROL SYSTEM

(71) Applicant: Gi-Shih LIEN, Taipei City (TW)

(72) Inventors: Gi-Shih LIEN, Taipei City (TW); Chih-Wen LIU, Taipei City (TW); Joe-Air JIANG, Taipei City (TW); Cheng-Long CHUANG, Taipei City (TW); Yu-Hao CHANG, Taipei City (TW); Wen-Chi HUANG, Taipei City (TW)

(21) Appl. No.: 14/484,809

(22) Filed: Sep. 12, 2014

(30) Foreign Application Priority Data
Sep. 26, 2013 (TW) 102134705

(51) Int. Cl. A61B 1/00; A61B 1/06; A61B 1/12; A61B 1/04

(52) U.S. Cl. CPC A61B 1/00158; A61B 1/041; A61B 1/00039; A61B 1/00029; A61B 1/00009; A61B 1/00016; A61B 1/0661; A61B 1/00045; A61B 1/00055; A61B 1/00006; A61B 1/128
USPC 600/102

(57) ABSTRACT

A capsule endoscope magnetic control system includes a control handle assembly, and a capsule endoscope having a controlled unit, a wireless power receiving unit, an image capturing unit, a processing unit, and a wireless communication unit. The control handle assembly includes a magnetic control unit for generating a magnetic field and a wireless power transmission unit for generating electromagnetic waves. After the capsule endoscope is placed inside a test object, the controlled unit fixed to the outside of the capsule endoscope changes the rotational direction of the capsule endoscope according to the changes in the magnetic field. The wireless power receiving unit generates an induced current. The image capturing unit generates an image data, which is received and converted by the processing unit into an image signal, which is transmitted by the wireless communication unit to the control handle assembly. Therefore, the capsule endoscope has a long battery life.

### FIELD OF THE INVENTION

The present invention relates to capsule endoscope techniques, and, more particularly, to a capsule endoscope magnetic control system that controls the state of the capsule endoscope through the use of an external handheld controller.

### BACKGROUND OF THE INVENTION

In the internal diagnosis and treatment of organisms, endoscope is a widely used tool. Early endoscopy involves installing a camera lens at the tip of a fiber-optic catheter of an endoscope, and inserting the camera lens and the fiber-optic catheter into the test object's mouth or anus. The camera captures the internal images of the test object and returns them back to an external device through the fiber-optic catheter. However, the stomach of a human body has many bends, and the camera lens is moved forward by pushing the fiber-optic catheter. This can cause discomfort in the test object during the examination process.

Later on, owing to the rapid development of medical equipment, early endoscopes with fiber-optic catheters are replaced by capsule endoscopes, which can be swallowed by the test object. Since there is no fiber-optic catheter, the test object does not feel discomfort. However, once the capsule endoscope is inside the test object, the movement of the capsule endoscope is achieved by gastrointestinal motility. In other words, the practitioner has no way of knowing the location of the capsule endoscope or moving it to a specific location. As a result, the capsule endoscopy may fail to obtain a desired image. Therefore, how to control the movement of a capsule endoscope has become a very important issue.

In recent years, large magnetic fields are created through instruments in order to guide the movements of the capsule endoscope and the image captured inside the test object. For example, a test object may lie on a large platform, and then the capsule endoscope is guided by moving a mechanical arm. However, such a device is bulky and costly and is limited by space when in use, and it is not easy and intuitive enough to use.

Furthermore, although the magnetically-controlled capsule endoscopy addresses some issues of the earlier endoscopy, there are still many problems to be overcome. First, the number of batteries that can be equipped in a capsule endoscope is limited by the size of the capsule endoscope, so the battery life is limited (usually eight hours), making it extremely inconvenient to use, since it usually takes up to two hours for a capsule endoscope to move to a desired shooting location by gastrointestinal motility after the endoscope is activated, and precisely when the endoscope reaches the desired location is difficult to calculate. Due to limited battery, the quantity and quality of the images captured are also under restrictions, that is, large number of high quality images cannot be provided to the practitioner. In addition, since the capsule endoscope and instrument are both implemented using permanent magnets, and therefore the size of the magnetic field created cannot be modified. If the attraction force is too large, the test object may feel discomfort since he/she can feel the presence of the capsule endoscope. On the other hand, if the attraction force is too small, insensitive control may occur.

Therefore, there is an urgent need to provide a control mechanism that addresses the issue of low internal power supply of the capsule endoscopes and provides effective capsule endoscopes.

### SUMMARY OF THE INVENTION

In light of the foregoing drawbacks, an objective of the present invention is to provide a capsule endoscope magnetic control system that improves the controllability and operations of the capsule endoscope by changing the magnetic control method and the addition of wireless power supply mechanism.

In accordance with the above and other objectives, the present invention provides a capsule endoscope magnetic control system, which may include a control handle assembly and a capsule endoscope. The control handle assembly may include a magnetic control unit for generating a magnetic field, and a wireless power transmission unit for generating electromagnetic waves. The capsule endoscope is to be placed inside a test object, and may include a controlled unit, a wireless power receiving unit, an image capturing unit, a processing unit, and a wireless communication unit. The controlled unit is fixed to the outside of the capsule endoscope for moving and turning the capsule endoscope according to a change in the magnetic field. The wireless power receiving unit is used for sensing the electromagnetic waves and generating an induced current through the electromagnetic waves. The image capturing unit is used for capturing a state of the test object to generate an image data. The processing unit is used for receiving the image data and converting the image data into an image signal. The wireless communication unit is used for transmitting the image signal to the control handle assembly. The induced current generated by the wireless power receiving unit is used for providing power required for the operations of the image capturing unit, the processing unit and the wireless communication unit.

In an embodiment, the magnetic control unit includes a plurality of electromagnets, and the controlled unit includes a plurality of permanent magnets.

In another embodiment, the control handle assembly further includes a wireless receiving unit for receiving the image signal transmitted by the wireless communication unit of the capsule endoscope. In addition, the wireless receiving unit determines a distance between the control handle assembly and the capsule endoscope based on the strength of the image signal received.

In yet another embodiment, the control handle assembly further includes a display for displaying the image signal received by the wireless receiving unit.

The capsule endoscope magnetic control system described in the present invention may further include a control-box device electrically connected to the control handle assembly. The control-box device may include a power supply unit, a control logic unit, and an inverter. The power supply unit is used for supplying power to drive the operations of the magnetic control unit of the control handle assembly. The control logic unit is used for receiving a control signal from a control button on the control handle assembly to generate a feedback signal. The inverter is used for modifying the power provided by the power supply unit according to the feedback signal generated by the control logic unit in order to change the magnetic field and the magnetic polarity of the magnetic control unit of the control handle assembly.

In another embodiment, the control-box device may further include a control interface for controlling the power provided by the power supply unit and displaying the image signal received by the control logic unit.

In yet another embodiment, the control handle assembly may further include a cooling unit for dissipating heat generated by the magnetic control unit. In further another embodiment, the control handle assembly may further include a plurality of phase radars for detecting the angle and the location of the capsule endoscope.

Furthermore, in a specific implementation, the capsule endoscope may be egg-shaped or droplet-shaped with a narrower top and a wider bottom to facilitate movement of the capsule endoscope inside the test object.

Compared to the prior art, the capsule endoscope magnetic control system according to the present invention controls the capsule endoscope through changing the magnetic field of the electromagnets, which can be controlled compared to traditional permanent magnets of which the magnetic field cannot be controlled. Moreover, power required for the internal operations of the capsule endoscope can be generated by electromagnetic induction between the control handle assembly of the present invention and the capsule endoscope, thus power is not constrained as in the prior art. Furthermore, a plurality of phase radars can be provided on the control handle assembly to overcome the shortcoming that the location of the capsule endoscope cannot be determined during use in the prior art. With the capsule endoscope magnetic control system of the present invention, failing to detect desired images or poor image quality as a result of power issue of the capsule endoscope can thus be resolved. Moreover, practitioners may intuitively control and examine the images captured by the endoscope through the handheld controller, providing great help for the gastrointestinal endoscopy.

### BRIEF DESCRIPTION OF THE DRAWINGS

The present invention can be more fully understood by reading the following detailed description of the preferred embodiments, with reference made to the accompanying drawings, wherein:

FIG. 1 is a schematic diagram depicting a capsule endoscope magnetic control system in accordance with the present invention;

FIG. 2 is a schematic diagram illustrating a specific implementation of the capsule endoscope magnetic control system in accordance with the present invention;

FIG. 3 is a schematic diagram illustrating a control handle assembly of the capsule endoscope magnetic control system in accordance with the present invention;

FIG. 4 is a schematic diagram illustrating a capsule endoscope of the magnetic control system in accordance with the present invention;

FIG. 5 is a schematic diagram illustrating the capsule endoscope magnetic control system in accordance with the present invention when in use;

FIG. 6 is a schematic diagram depicting different arrangements of the electromagnets of the capsule endoscope magnetic control system in accordance with the present invention;

FIG. 7 is a schematic diagram illustrating another embodiment of the control handle assembly of the capsule endoscope magnetic control system in accordance with the present invention; and

FIGS. 8A and 8B are schematic diagrams illustrating different shapes of the capsule endoscope of the capsule endoscope magnetic control system in accordance with the present invention.

### DETAILED DESCRIPTION OF THE EMBODIMENTS

The present invention is described by the following specific embodiments. Those with ordinary skills in the arts can readily understand other advantages and functions of the present invention after reading the disclosure of this specification.

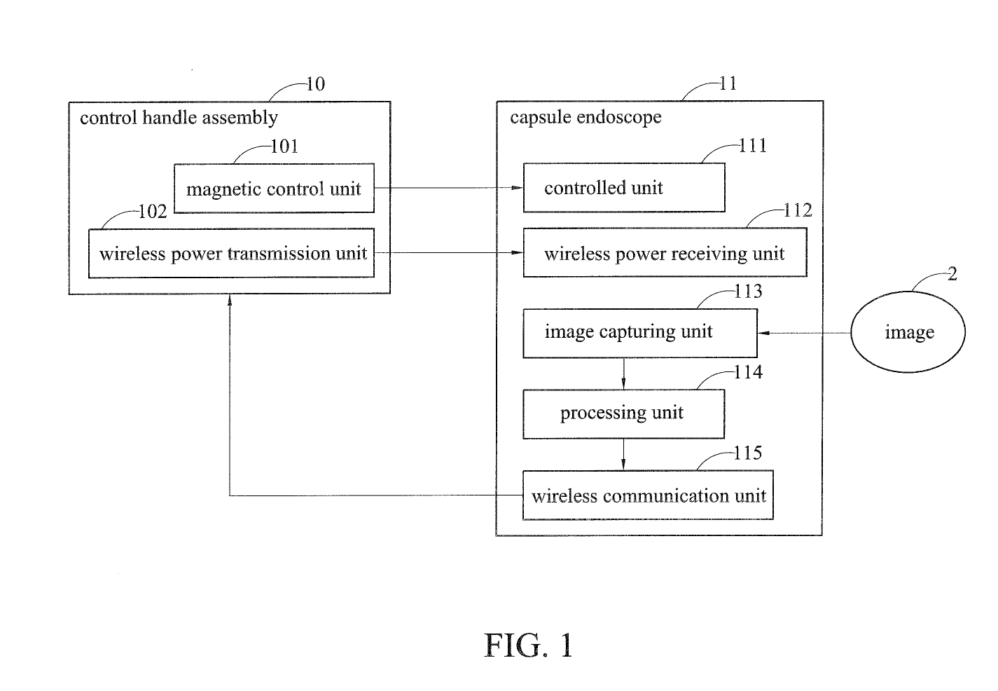

Referring to FIG. 1, a schematic diagram depicting a capsule endoscope magnetic control system 1 in accordance with the present invention is shown. The capsule endoscope magnetic control system 1 includes a control handle assembly 10 and a capsule endoscope 11. The capsule endoscope 11 is to be swallowed by a test object, and a practitioner controls the capsule endoscope 11 through the use of the control handle assembly 10 in order to move the capsule endoscope 11 to a desired location and at an appropriate angle for image capturing.

The control handle assembly 10 includes a magnetic control unit 101 and a wireless power transmission unit 102. The magnetic control unit 101 is used for generating a magnetic field to control the capsule endoscope 1. In an embodiment, the control handle assembly 10 may be equipped with a driving motor for driving the magnetic control unit 101 to rotate, and in turn controlling the capsule endoscope 11. In addition, the wireless power transmission unit 102 is used for generating electromagnetic waves to allow the capsule endoscope 11 to generate electrical energy through the induction principle.

The capsule endoscope 11 is to be placed in the test object. The capsule endoscope 11 includes a controlled unit 111, a wireless power receiving unit 112, an image capturing unit 113, a processing unit 114, and a wireless communication unit 115.

The controlled unit 111 is fixed to the outside of the capsule endoscope 11. The capsule endoscope 11 is moved or rotated by the controlled unit 111 according to the variations in the magnetic field created by the magnetic control unit 101. More specifically, by changing the magnetic field created by the control handle assembly 10, the controlled unit 111 is moved or rotated accordingly under the magnetic concept; that is, same poles attract each other and opposite poles repel each other.

Additionally, in order to avoid the shortcoming of an unchangeable magnetic field created by permanent magnets at the controlling end and the endoscope end, the magnetic control unit 101 comprises a plurality of electromagnets, and the controlled unit 111 comprises a plurality of permanent magnets. In other words, the magnetic control unit 101 can modify the size of its magnetic field to eliminate attraction force being too small or too large.

The wireless power receiving unit 112 is used for sensing the electromagnetic waves generated by the wireless power transmission unit 102 and inducing a current from the electromagnetic waves through electromagnetic induction. More specifically, the provision of the wireless power transmission unit 102 of the control handle assembly 10 and the wireless power receiving unit 112 of the capsule endoscope 11 addresses the issue of a short battery life of the capsule endoscope 11. In this embodiment, electromagnetic waves are transmitted by the wireless power transmission unit 102. Once sensing the electromagnetic waves, the wireless power receiving unit 112 induces a current through changes in the magnetic field, and the induced current can be used by the capsule endoscope 11.

The image capturing unit 113 is used for capturing images 2 inside the test object and generating image data. Since the capsule endoscope 11 is used for photographing the test object, the image capturing unit 113 is provided in the capsule endoscope 11. In this embodiment, in order to gain a more complete picture, the capsule endoscope 11 is provided with two camera lenses, one located in front of the capsule endoscope 11, and the other located at the side of the capsule endoscope 11, to help capturing a more complete image.

The processing unit 114 is used for receiving the image data captured by the image capturing unit 113 and converting the image data into image signals. The processing unit 114 may be a typical microcontroller that is capable of performing a variety of functions, such as calculating, storing information or inputting/outputting, and its functions will not be further described as it is a well-known component in this art.

The wireless communication unit 115 is used for sending the image signals generated by the processing unit 114 to the control handle assembly 10. In this embodiment, the capsule endoscope 11 sends the image signals obtained in real time through the wireless communication unit 115, without storing the signals internally. As such, image quality can be improved and shortage of storage is not an issue. In addition, the control handle assembly 10 and the capsule endoscope 11 communicate with each other through wireless transmission, eliminating the inconvenience of the early endoscope equipped with fiber-optic catheter.

From the above, in order to obtain complete images of a test object, the capsule endoscope 11 starts to take images once it enters into the test object, so it will need adequate power. Furthermore, the wireless communication unit 115 will transmit image signals to the control handle assembly 10 in real time; it also needs to consume a large amount of power. Therefore, in the present embodiment, the induced current is generated by the wireless power receiving unit 112 to provide power needed by the image capturing unit 113, the processing unit 114, the wireless communication unit 115 or other built-in components in the capsule endoscope 11 during operation, thereby addressing the problems of the prior-art capsule endoscopes.

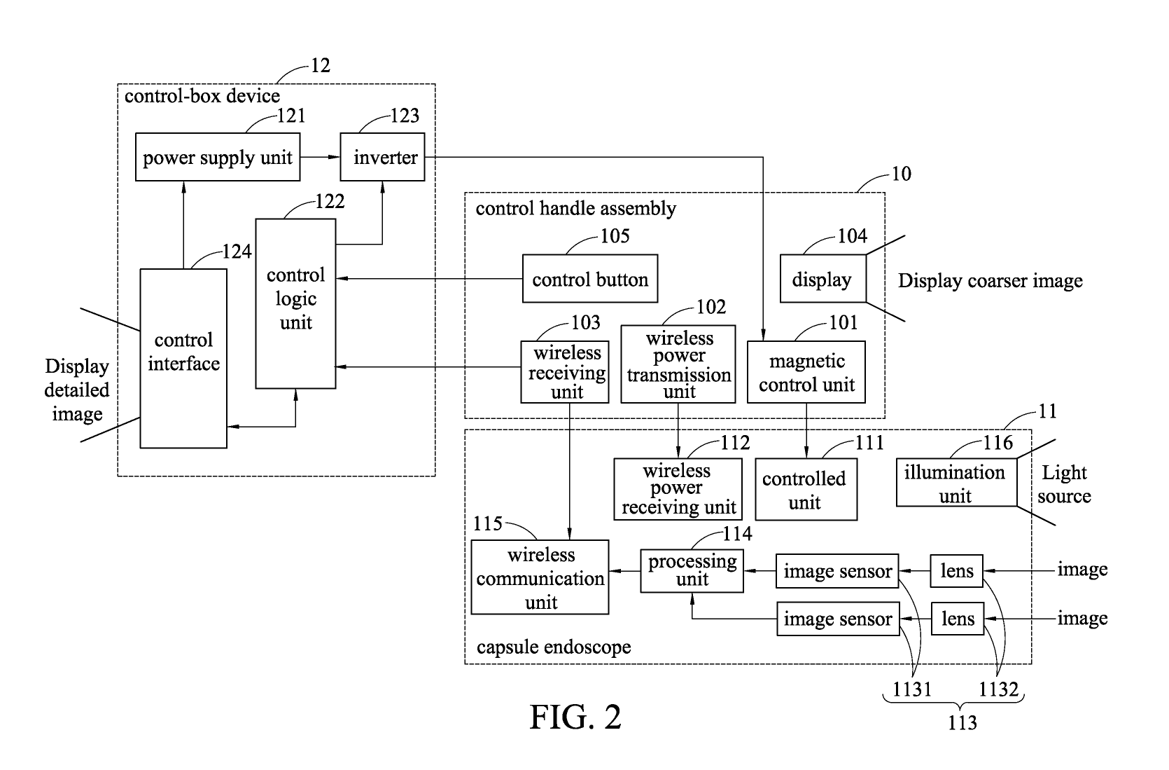

Referring to FIG. 2, a schematic diagram illustrating a specific implementation of the capsule endoscope magnetic control system in accordance with the present invention is shown. As shown, in order to overcome the inconvenience of using the magnetic field of a bulky instrument to control the capsule endoscope, the present invention proposes the control handle assembly 10 to allow easy manipulation by the practitioner. The control handle assembly 10 is designed like a handle. In order to reduce the weight and volume of the control handle assembly 10, in this embodiment, a control-box device 12 electrically connected with the control handle assembly 10 is provided in the capsule endoscope magnetic control system 1. In this way, heavier or non-critical elements can be located in the control-box device 12, so that the practitioner can easily manipulate the control handle assembly 10.

The control-box device 12 can be connected to the control handle assembly 10 through a transmission line. In one example, the control-box device 12 includes a power supply unit 121, a control logic unit 122, and an inverter 123.

The power supply unit 121 is used for providing power in order to drive the magnetic control unit 101 in the control handle assembly 10. As mentioned earlier, the magnetic control unit 101 comprises a plurality of electromagnets. A magnetic field is created or changes in the magnetic field are created in the magnetic control unit 101 through the power provided by the power supply unit 121.

The control logic unit 122 is used for receiving control signals sent from control buttons 105 on the control handle assembly 10 to generate a feedback signal. More specifically, the practitioner manipulates the control handle assembly 10 through the control buttons 105. For example, the practitioner manipulates the driving motor in the control handle assembly 10 to rotate clockwise or anticlockwise, thereby changing the magnetic field of the magnetic control unit 101. Thus, the control logic unit 122 will provide a feedback signal according to the control signals of the control buttons 105 to change the magnetic field of the magnetic control unit 101.

The inverter 123 is used for modifying the power provided by the power supply unit 121 according to the feedback signal of the control logic unit 122 in order to change the magnetic field and the magnetic polarity of the magnetic control unit 101 of the control handle assembly 10. As mentioned earlier, when the driving motor in the control handle assembly 10 rotates clockwise or anticlockwise, the magnetic field of the magnetic control unit 101 is changed, which in turn rotates or moves the controlled unit 111 of the capsule endoscope 11 as a result of changes in the magnetic field of the magnetic control unit 101.

Moreover, the control-box device 12 further includes a control interface 124 for controlling the power provided by the power supply unit 121 and displaying the image signals received by the control logic unit 122 from the control handle assembly 10. In an example, the control interface 124 and the power supply unit 121 can transmit signals between each other through a Universal Serial Bus (USB). The control interface 124 and the control logic unit 122 can be connected through an RS232 interface, and the control interface 124 may display real-time images in addition to controlling the power provided by the power supply unit 121.

The control handle assembly 10 includes, in addition to the magnetic control unit 101 and the wireless power transmission unit 102, a wireless receiving unit 103, a display 104, and the control buttons 105. The wireless receiving unit 103 is used for receiving the image signals transmitted by the wireless communication unit 115 of the capsule endoscope 11. Since there is no physical link between the control handle assembly 10 and the capsule endoscope 11, the image signals transmitted by the wireless communication unit 115 is received by the wireless receiving unit 103 of the control handle assembly 10, and the image signals received by the wireless receiving unit 103 can be not only displayed by the display 104, but also returned back to the control-box device 12.

The purpose of the display 104 is to enable the practitioner to observe images during the manipulation process in order to manipulate the movement or rotation of the control handle assembly 10. However, in order to reduce the volume and weight of the control handle assembly 10, the display 104 only displays coarser images. If finer images are needed, they are available on the control interface 124 of the control-box device 12. In addition, the control buttons 105 allow the practitioner to easily change the magnetic field of the magnetic control unit 101 during operation.

The capsule endoscope 11 includes, in addition to the controlled unit 111, the wireless power receiving unit 112, the image capturing unit 113, the processing unit 114 and the wireless communication unit 115, an illumination unit 116. The illumination unit 116 may be a light emitting diode for providing light required for the image capturing unit 113 to take images when the capsule endoscope 11 is inside the test object. Moreover, the image capturing unit 113 can comprise an image sensor 1131 and a lens 1132, for example an image sensor 1131 equipped with a CMOS lens.

In addition, in order for the practitioner to have an idea of the distance between the control handle assembly 10 and the capsule endoscope 11, the present invention further proposes that the strength of the image signals received by the wireless receiving unit 103 be determined in order to know the distance between the control handle assembly 10 and the capsule endoscope 11. During wireless transmission, the distance affects the strength of the wireless signals, so the control handle assembly 10 can determine the distance between the control handle assembly 10 and the capsule endoscope 11 by determining the strength of image signals received by the wireless receiving unit 103. This helps the practitioner to manipulate and reduces discomfort caused by large attraction force.

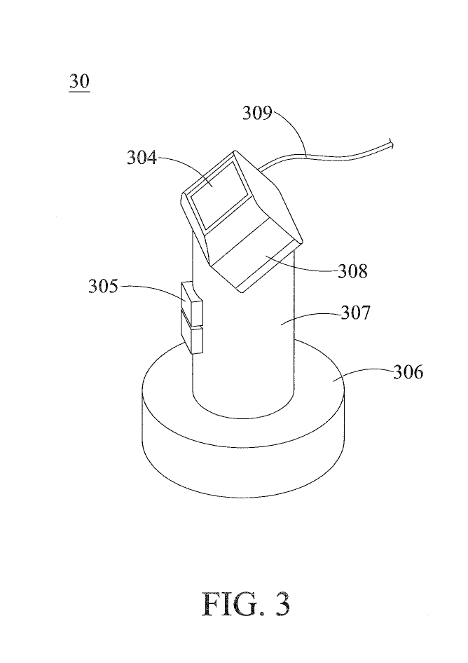

Referring to FIG. 3, a schematic diagram illustrating a control handle assembly of the capsule endoscope magnetic control system in accordance with the present invention is shown. In conjunction to FIG. 2, a control handle assembly 30 mainly includes a front end portion 306, a grip portion 307, a rear end portion 308, and a connection cable 309. The practitioner may hold the grip portion 307 during operation. Control buttons 305 on the grip portion 307 allow the driving motor in the control handle assembly 30 to change its rotation direction. The rear end portion 308 can be provided with a display 304 for displaying images to the practitioner during operation.

Moreover, the rear end portion 308 can return images through the connection cable 309 to the control-box device 12 of FIG. 2, or the control-box device 12 can provide power to the control handle assembly 30 through the connection cable 309. In addition, the front end portion 306 allows the magnetic control unit 101 of FIG. 2 to be provided therein, so the practitioner controls the capsule endoscope 11 of FIG. 2 by changing the magnetic field of the magnetic control unit 101 in the front end portion 306.

In other embodiments, the control handle assembly 30 further includes a cooling unit (not shown) that can be provided in the front end portion 306 for cooling the heat generated by the magnetic control unit 101. The magnetic control unit 101 having electromagnets will generate heat energy during operation, and the heat is dissipated through a liquid such as oil. Furthermore, the liquid may be brought back to the control-box device 12 and heat dissipation is achieved through a fan (not shown) in the control-box device 12.

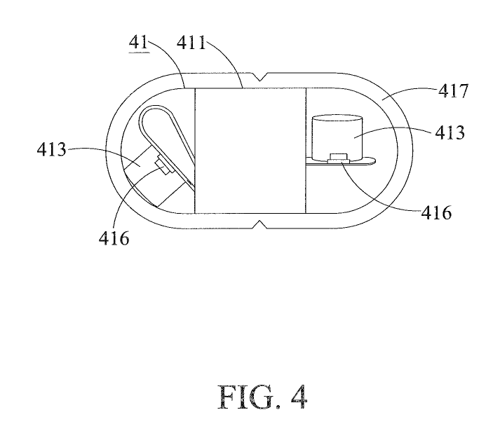

Referring to FIG. 4, a schematic diagram illustrating a capsule endoscope of the capsule endoscope magnetic control system in accordance with the present invention is shown. As shown, a capsule endoscope 41 is encapsulated by a transparent optical cover 417 to protect the elements in the capsule endoscope 41 from being damaged inside the test object. The capsule endoscope 41 includes a controlled unit 411 having permanent magnets, and image capturing units 413 facing two different directions for taking images from different angles and an illumination unit 416. The wireless power receiving unit 112, the processing unit 114 and the wireless communication unit 115 described with respect to FIG. 2 can be provided on a single chip inserted into the capsule endoscope 41.

In an embodiment, the controlled unit 411 surrounds the periphery of the capsule endoscope 41. It has a plurality of raised parts such that, when the controlled unit 411 rotates as a result of changes in magnetic field of the magnetic control unit 101 of FIG. 2, the capsule endoscope 41 is propelled by these raised parts.

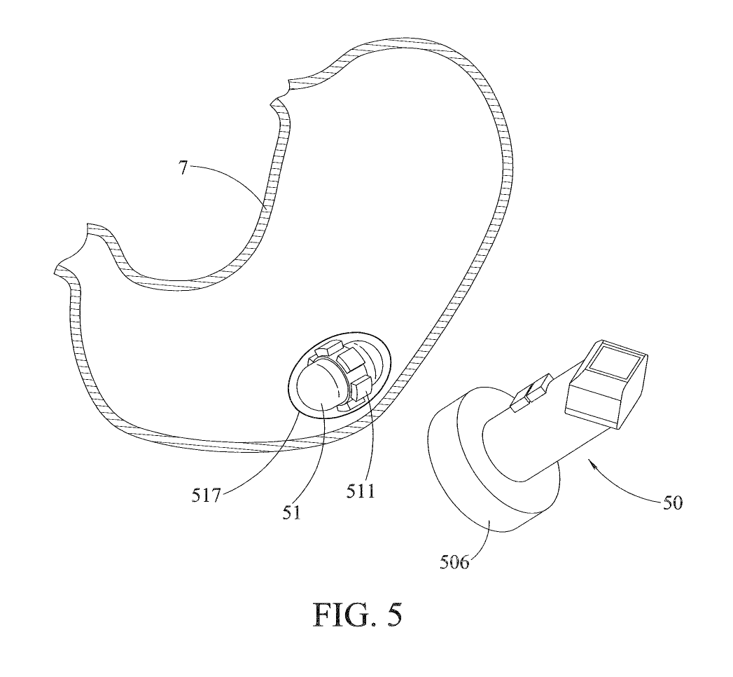

Referring to FIG. 5, a schematic diagram illustrating the capsule endoscope magnetic control system in accordance with the present invention when in use is shown. After a capsule endoscope 51 (protected by an optical cover 517) is swallowed by a test object and reaches an organ 7 of the test object, the practitioner then manipulates the location and/or angle of the capsule endoscope 51 through a control handle assembly 50. That is, through magnetic changes of the electromagnets in a front end portion 506 of the control handle assembly 50, a controlled unit 511 of the capsule endoscope 51 is changed accordingly, i.e., the capsule endoscope 51 rotates around its shaft to change the angle of the shooting lens, or the controlled unit 511 rotates around the capsule endoscope 51 to change the direction of progression of the capsule endoscope 51. The practitioner can look at the images directly from a display of the control handle assembly 50. Regarding the electromagnets in the front end portion 506 of the control handle assembly 50, they can be arranged in different ways to achieve different magnetic controls.

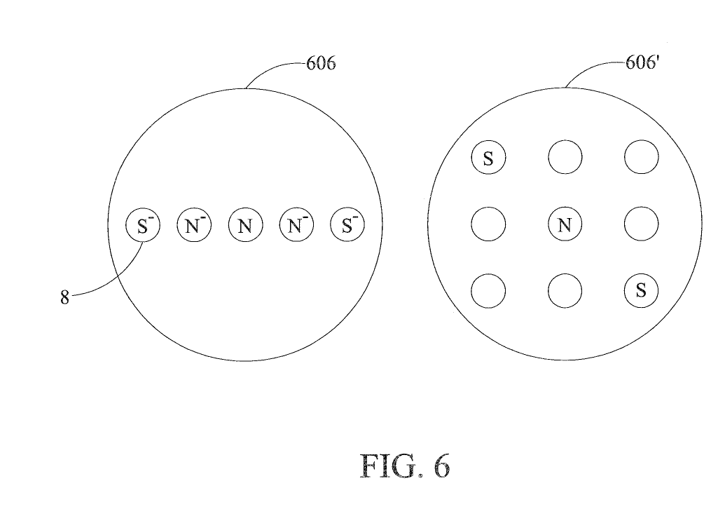

Referring to FIG. 6, a schematic diagram depicting different arrangements of the electromagnets of the capsule endoscope magnetic control system in accordance with the present invention is shown. In the left hand side of FIG. 6, a front end portion 606 of the control handle assembly is provided with several electromagnets 8 arranged in a one-dimensional manner, for example, their poles are in the following order: weak S pole, weak N pole, strong N pole, weak N pole, and weak S pole. When the front end portion 606 rotates as a result of the driving motor, the rotational direction of the capsule endoscope 51 shown in FIG. 5 will be affected, allowing control of the movement of the capsule endoscope 51. On the other hand, in the right hand side of FIG. 6, a front end portion 606′ of the control handle assembly is also provided with several electromagnets 8 arranged in a two-dimensional manner. When the front end portion 606′ rotates as a result of the driving motor, the rotational direction of the capsule endoscope 51 will be affected, also allowing control of the movement and direction of the capsule endoscope 51.

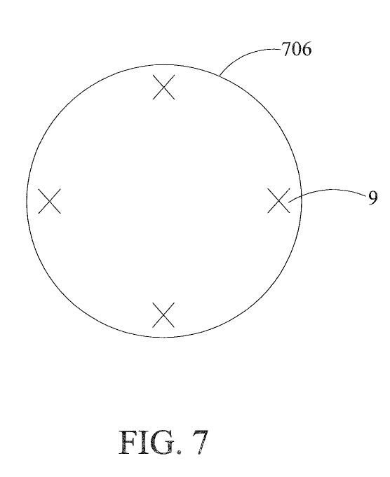

Referring to FIG. 7, a schematic diagram illustrating another embodiment of the control handle assembly of the capsule endoscope magnetic control system in accordance with the present invention is shown. When the capsule endoscope is inside the test object, in order for the practitioner to readily know where the capsule endoscope is, the control handle assembly further includes a plurality of phase radars 9 for detecting the angle and the location of the capsule endoscope. As shown, the plurality of phase radars 9 are provided in a front end portion 706 of the control handle assembly for detecting the angle and the location of the capsule endoscope, thereby assisting the practitioner to more quickly find out where the capsule endoscope is.

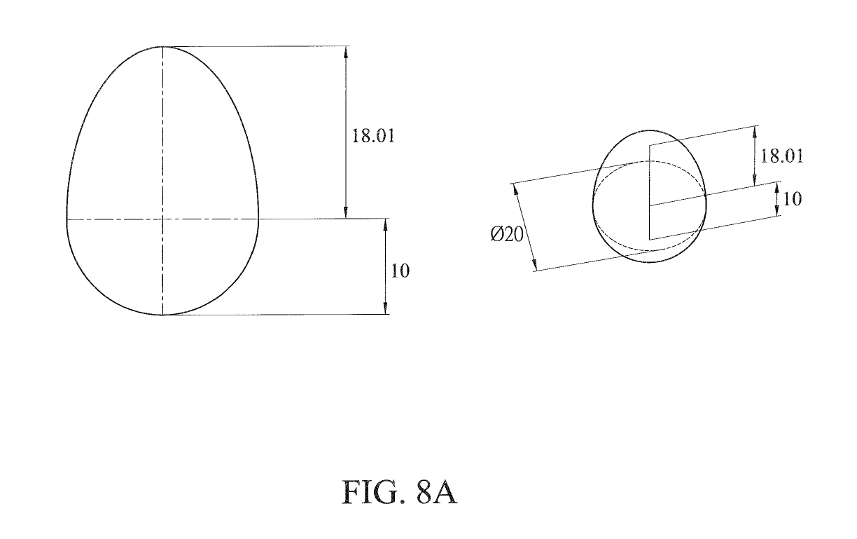

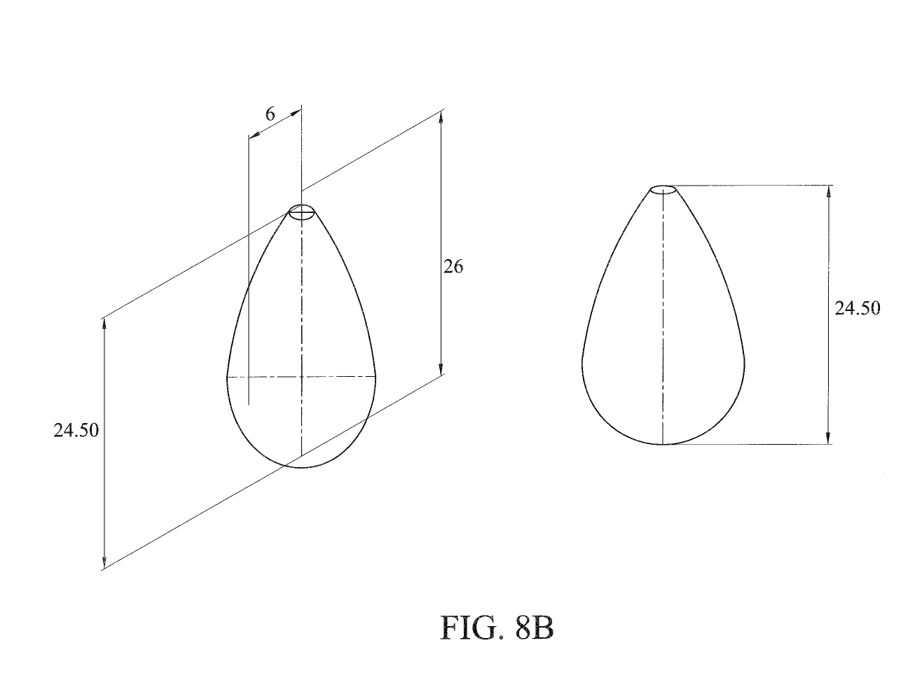

FIGS. 8A and 8B are schematic diagrams illustrating different shapes of the capsule endoscope of the magnetic control system in accordance with the present invention. The capsule endoscope proposed by the present invention is moved forward by the esophagus muscle in the esophagus. A conventional capsule endoscope usually takes the form of a capsule. Since its shape is similar to a cylindrical shape, this adversely affects the movement of the capsule endoscope in the esophagus. In this regard, the present invention further proposes that the capsule endoscope to be designed to be narrower towards the top and wider towards the bottom. After experiments in various angles, the angle of the tapered end of the capsule endoscope is preferably about 30°, that is, the opening angle of the narrower end is about 30°.

In FIG. 8A, the capsule endoscope is designed to have an egg-shaped appearance. As shown, it can be designed with a maximum diameter of 20 mm. An intersection is specified as the intersection between the maximum vertical tangent line and the maximum diameter tangent line, and the distance between this intersection and the tip at the narrower end is 18.01 mm, while the distance between the intersection and the tip at the wider end is 10 mm. In actual use, the wider end can be swallowed first. This will facilitate the movement of the capsule endoscope in the esophagus.

In addition, in FIG. 8B, the capsule endoscope is designed to have a water droplet shape. As shown, the maximum diameter is 12 mm. In terms of the appearance of water droplet, the maximum vertical distance can be 26 mm, but taking into consideration that a sharp end is hazardous, so the sharp structure at the narrower end is truncated, so yielding an overall maximum vertical distance of 24.05 mm. Similarly, in actual use, the wider end can be swallowed first. This will facilitate the movement of the capsule endoscope in the esophagus.

It should be noted that, regardless of a egg-shaped or a droplet-shaped design, the upper end (i.e., the narrower end) of the capsule endoscope can be wired or wireless, and the upper and lower ends needs to be rounded to have an overall smooth appearance to avoid damaging the wall of the gastrointestinal tract during examination. With the design of a tapered shape, the stress exerted upon the capsule endoscope when the esophagus contracts will be maximized, facilitating the movement of the capsule endoscope in the human body.

In summary, the capsule endoscope magnetic control system proposed by the present invention controls the capsule endoscope through changing the magnetic field of the electromagnets, which can be controlled compared to traditional permanent magnets of which the magnetic field cannot be controlled. Moreover, power required for the internal operations of the capsule endoscope can be generated by electromagnetic induction between the control handle assembly of the present invention and the capsule endoscope. This indirectly solves the problem that images cannot be taken throughout the whole process and image transmission issue due to a lack of power in the capsule endoscope. With the capsule endoscope magnetic control system of the present invention, power issue of the capsule endoscope can be easily solved, and practitioners may intuitively control and examine the images captured by the endoscope through the handheld controller, providing great help for the gastrointestinal endoscopy.

The above embodiments are only used to illustrate the principles of the present invention, and should not be construed as to limit the present invention in any way. The above embodiments can be modified by those with ordinary skill in the art without departing from the scope of the present invention as defined in the following appended claims.

What is claimed is:

1. A capsule endoscope magnetic control system, comprising:
a control handle assembly including a magnetic control unit for generating a magnetic field and a wireless power transmission unit for generating electromagnetic waves; and
a capsule endoscope to be placed inside a test object, including:
a controlled unit fixed to the outside of the capsule endoscope for moving and turning the capsule endoscope according to a change in the magnetic field;
a wireless power receiving unit for sensing the electromagnetic waves and generating an induced current through the electromagnetic waves;
an image capturing unit for capturing a state of the test object to generate an image data;
a processing unit for receiving the image data and converting the image data into an image signal; and
a wireless communication unit for transmitting the image signal to the control handle assembly,
wherein the induced current generated by the wireless power receiving unit is used for providing power required for the operations of the image capturing unit, the processing unit and the wireless communication unit.

2. The capsule endoscope magnetic control system claim 1, wherein the magnetic control unit includes a plurality of electromagnets, and the controlled unit includes a plurality of permanent magnets.

3. The capsule endoscope magnetic control system of claim 1, wherein the image capturing unit includes an illumination unit for providing a light source.

4. The capsule endoscope magnetic control system of claim 1, wherein the control handle assembly further includes a wireless receiving unit for receiving the image signal transmitted by the wireless communication unit of the capsule endoscope.

5. The capsule endoscope magnetic control system of claim 4, wherein the wireless receiving unit determines a distance between the control handle assembly and the capsule endoscope based on the strength of the image signal received.

6. The capsule endoscope magnetic control system of claim 4, wherein the control handle assembly further includes a display for displaying the image signal received by the wireless receiving unit.

7. The capsule endoscope magnetic control system of claim 1, further comprising a control-box device electrically connected to the control handle assembly, the control-box device including:
a power supply unit for supplying power to drive the operations of the magnetic control unit of the control handle assembly;
a control logic unit for receiving a control signal from a control button on the control handle assembly to generate a feedback signal; and
an inverter for modifying the power provided by the power supply unit according to the feedback signal generated by the control logic unit to change the magnetic field and magnetic polarity of the magnetic control unit of the control handle assembly.

8. The capsule endoscope magnetic control system of claim 7, wherein the control-box device further includes a control interface for controlling the power provided by the power supply unit and displaying the image signal received by the control logic unit.

9. The capsule endoscope magnetic control system of claim 1, wherein the control handle assembly further includes a cooling unit for dissipating heat generated by the magnetic control unit.

10. The capsule endoscope magnetic control system of claim 1, wherein the control handle assembly further includes a plurality of phase radars for detecting an angle and location of the capsule endoscope.

11. The capsule endoscope magnetic control system of claim 1, wherein the capsule endoscope is egg-shaped or droplet-shaped with a narrower top and a wider bottom.
